(19) United States
(12) Patent Application Publication
(10) Pub. No.: US 2009/0275655 A1
(43) Pub. Date: Nov. 5, 2009

(54) Pharmaceutical Gallium Compositions and Methods

(75) Inventors: Raymond P. Warrell, JR., Westfield, NJ (US); Bob D. Brown, Millington, NJ (US)

Correspondence Address: DIEHL SERVILLA LLC, 77 BRANT AVE, SUITE 210, CLARK, NJ, 07066, US

(73) Assignee: Genta Incorporated, Berkeley Heights, NJ (US)

(21) Appl. No.: 12/431,058

(22) Filed: Apr. 28, 2009

Related U.S. Application Data
(60) Provisional application No. 61/110,674, filed on Nov. 3, 2008.
(60) Provisional application No. 61/049,017, filed on Apr. 30, 2008.

(51) Int. Cl. A61K 31/28

(52) U.S. Cl. 514/492

(57) ABSTRACT

The present invention provides novel pharmaceutical gallium compositions, as well as methods for their preparation and methods for treating conditions and diseases such as cancer, hypercalcemia, osteoporosis, osteopenia, Paget's disease, and infections.

### FIELD OF THE INVENTION

The present invention relates generally to pharmaceutical gallium compositions, in particular those suitable for therapeutic oral or intravenous administration.

### BACKGROUND OF THE INVENTION

Gallium has demonstrated pharmaceutical value for the treatment of many human and animal disorders, including hypercalcemia, cancer, metastatic bone disease, infections, and especially certain widespread degenerative or metabolic bone diseases such as osteoporosis and Paget's disease. For example, numerous clinical studies have shown gallium to have antineoplastic activity, as well as the ability to reduce abnormally high bone turnover in Paget's disease (reviewed in Bernstein, Therapeutic Gallium Compounds, in Metallotherapeutic Drugs and Metal-Based Diagnostic Agents: The Use of Metals in Medicine 259-277 (Gielen and Tiekink eds., 2005)). Gallium is currently approved for use in the United States as a gallium nitrate (Ga(NO₃)₃.9H₂O) solution for intravenous infusion (Ganite®) to treat hypercalcemia of malignancy.

In spite of its established utility, however, the use of gallium in the treatment of such diseases is hampered by the fact that simple forms of gallium, such as gallium salts and organometallic complexes, are very poorly absorbed when delivered orally. The low oral bioavailability of these gallium forms requires that either impractically large doses of orally delivered gallium be administered to the patient or that the gallium be administered via non-oral means (e.g., intravenous delivery). At present, the repeated or chronic administration via the oral route of such gallium salts, in particular the chloride (halogen), nitrate, sulfate, etc. salts, is not believed to be practical with chronic conditions such as osteoporosis and Paget's disease due to their low bioavailability, lack of pharmaceutical acceptability or both.

Efforts have been made to increase the bioavailability of orally administered gallium, particularly through chemical complexing. Several gallium complexes have been identified that demonstrate increased oral bioavailability, including, e.g., gallium maltolate (see, e.g., Bernstein et al., Metal-Based Drugs 7:33-47 (2000); U.S. Pat. Nos. 5,258,376; 5,574,027; 5,883,088; 5,968,922; 5,998,397; 6,004,951; 6,048,851; 6,087,354)) and gallium 8-quinolinolate (see, e.g., Collery et al., Anticancer Res. 16:687-692 (1996); U.S. Pat. No. 5,525,598; European Patent No. EP 0 599 881; International Application No. PCT/EP92/01687). Other therapeutic gallium complexes are described in, e.g., Arion et al., J. Inorg. Biochem. 91:298-305 (2002); Chitambar et al., Clin. Cancer Res. 2:1009-1015 (1996); Stojilkovic et al., Mol. Microbiol. 31:429-442 (1999); U.S. Pat. Nos. 5,196,412; 5,281,578; and International Application No. PCTJUS91/03599.

In this regard, the results of a recent Phase I study of a tablet formulation of the active ionic gallium ingredient contained in Ganite®, using the nitrate counter-ion complexed with a carrier excipient showed good absorption following oral administration in healthy volunteers. Nitrates, however, are not preferred as bacteria in the oral cavity and alimentary tract can reduce nitrates to nitrites, which have been implicated as risk factors predisposing to development of certain ailments, in particular forms of cancer. In addition, nitrates can induce serious drops in blood pressure (i.e., hypotension), both alone and in combination with certain drugs (e.g., sildenafil).

Thus, there is a continuing need for the development of new pharmaceutical gallium compositions, particularly those having enhanced oral bioavailablity and/or low toxicity.

### SUMMARY OF THE INVENTION

Embodiments of the present invention provide novel pharmaceutical gallium compositions and methods for their preparation, as well as methods for treating conditions and diseases in which inhibition of abnormally increased bone resorption or certain infections is desired. More particularly, embodiments of the present invention provide novel gallium citrate preparations suitable for oral administration and methods for its preparation, as well as methods for treating conditions and diseases such as cancer, hypercalcemia, osteoporosis, osteopenia, and Paget's disease (i.e., any bone disorder mediated by the action of osteoclasts and osteoblasts or tumor cells), as well as infections.

Accordingly, one aspect of the present invention is directed to a method for replacing one or more anionic counterions of a gallium salt with one or more citrate ions comprising reacting the gallium salt with a citrate salt in an aqueous solution, and recovering the resulting gallium citrate in which one or more anionic counterions have been replaced with one or more citrate ions. The gallium is preferably at a concentration of about 0. 1 M to saturation, more preferably about 0.75-1.25 M, most preferably about 1. 0 M, while the citrate is preferably at a concentration of about 0.1 M to saturation, more preferably about 0.5-4 M, most preferably about 2-3 M. The recovered gallium citrate can be washed with simple pharmaceutical solvents, pure water, or mixtures thereof. Preferably, no neutralization step is included prior to recovery of the gallium citrate.

The gallium salt may be any pharmaceutically acceptable gallium salt, including, but not limited to, gallium iodide, gallium chloride, gallium sulfate and gallium nitrate. Preferably, the starting gallium salt is gallium nitrate. Similarly, the citrate salt may be any pharmaceutically acceptable citrate salt, including, but not limited to, ammonium citrate, potassium citrate and sodium citrate.

Depending on the gallium salt and citrate salt used, the resulting gallium citrate may have low amounts of one or more pharmaceutically acceptable anions and/or cations bonded thereto. As such, the recovered gallium citrate will have the general formula I:

[Cat]ₓGa(Cit)_{y}[An]_{z}.nH₂O I

wherein [Cat] is a pharmaceutically acceptable cation, (Cit) is citrate wherein 0-3 of the carboxyl groups are optionally protonated, [An] is a pharmaceutically acceptable anion, x is 0-2, y is 1-6, z is 0-2, and n is 0-2 or n is 0-6. In a preferred embodiment, x is 0, y is 2, z is 0 and n is 0.

In a preferred embodiment, the starting gallium salt in the preparative method is gallium nitrate, and the citrate salt is ammonium citrate. Accordingly, the recovered gallium citrate will have the general formula II:

[NH₄]ₓGa(Cit)_{y}[NO₃]_{z}.nH₂O II

wherein (Cit) is as above, x is 0-2, y is 1-6, z is 0-2, and n is 0-2 or n is 0-6. In a preferred embodiment, x is 0, y is 2, z is 0 and n is 0.

Applicants have found that the gallium citrate made by the above method has lower amounts of undesirable complexed ions, such as ammonium, sodium, potassium, and nitrate ions, compared to prior art gallium citrate preparations, and thus is more appropriate for administration to living organisms. Accordingly, another aspect of the present invention is directed to pharmaceutical grade ammonium citrate. In a preferred embodiment, the pharmaceutical grade gallium citrate has the formula Ga(C₆H₅O₇)₂.

Another aspect of the present invention is directed to a pharmaceutical composition for administering gallium to a patient in need thereof comprising gallium citrate and one or more pharmaceutically acceptable excipients, wherein the gallium citrate has the general formula I:

[Cat]ₓGa(Cit)_{y}[An]_{z}.nH₂O I

wherein [Cat] is a pharmaceutically acceptable cation, (Cit) is citrate wherein 0-3 of the carboxyl groups are optionally protonated, [An] is a pharmaceutically acceptable anion, x is 0-2, y is 1-6, z is 0-2, and n is 0-2 or n is 0-6. In a preferred embodiment, the gallium citrate has the general formula II:

[NH₄]ₓGa(Cit)_{y}[NO₃]_{z}.nH₂O II

wherein (Cit) is as above, x is 0, y is 2, z is 0 and n is 0. In preferred embodiments, the composition is designed for oral, buccal, sublingual, or intranasal administration. In some embodiments, a complexing agent capable of complexing gallium is present in the composition. Because the gallium compounds of the present invention have improved bioavailability, the amount of complexing agents required may be reduced as compared to Ga(NO₃)₃ compounds.

Another aspect of the present invention is directed to a method for administering gallium to a patient in need thereof comprising administering to said patient a pharmaceutical composition comprising a therapeutically effective amount of gallium citrate and one or more pharmaceutically acceptable excipients, wherein the gallium citrate has the general formula I:

[Cat]ₓGa(Cit)_{y}[An]_{z}.nH₂O I

wherein [Cat] is a pharmaceutically acceptable cation, (Cit) is citrate wherein 0-3 of the carboxyl groups are optionally protonated, [An] is a pharmaceutically acceptable anion, x is 0-2, y is 1-6, z is 0-2, and n is 0-2 or n is 0-6. In a preferred embodiment, the gallium citrate has the general formula II:

[NH₄]ₓGa(Cit)_{y}[NO₃]_{z}.nH₂O II

wherein (Cit) is as above, x is 0, y is 2, z is 0 and n is 0. In some embodiments, a complexing agent capable of complexing gallium is present in the composition. In preferred embodiments, the composition is delivered by oral, buccal, sublingual, intranasal, or transdermal administration to a patient in need of treatment for a condition or disease characterized by excessive bone resorption.

Another aspect of the present invention is directed to a method of treating a patient having a bacterial infection comprising systemically administering to said patient an ionic gallium-containing compound in an amount sufficient to treat said infection. The infection can result from either gram-negative or gram-positive bacteria. Suitable ionic-gallium containing compounds include gallium nitrate and gallium citrate. The ionic-gallium containing compound can be systemically administered by any route that achieves an antibacterial concentration of ionic gallium, including, but not limited to, intravenous, subcutaneous, topical, oral, buccal, sublingual, or intranasal routes. Such administration can also be used to treat specific infectious conditions, such as, e.g., sepsis, pneumonia, wound infections, osteomyelitis, etc.

### BRIEF DESCRIPTION OF THE DRAWINGS

FIG. 1 is a graphic depiction of the bioavailability of two batches of gallium citrate prepared according to the present invention and a clinical prototype of Ga(NO₃)₃.9H₂O (gallium nitrate, used as the active pharmaceutical ingredient in Ganite®) following oral administration to dogs.

### DETAILED DESCRIPTION OF THE INVENTION

Although the invention herein is described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

The present invention relates to a gallium citrate preparation suitable for oral administration, as well as methods for its preparation and methods of treating conditions and diseases such as cancer, hypercalcemia, osteoporosis, osteopenia, Paget's disease, and infections.

“Patient” includes both human and other mammals. “Mammal” means humans and other mammalian animals.

The term “treating” or “treatment” of a state, disorder, disease or condition as used herein means: (1) preventing or delaying the appearance of clinical symptoms of the state, disorder, disease or condition developing in a mammal that may be afflicted with or predisposed to the state, disorder, disease or condition but does not yet experience or display clinical or subclinical symptoms of the state, disorder or condition, (2) inhibiting the state, disorder, disease or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient and/or to the physician.

“Effective amount” and “therapeutically effective amount” mean the amount of a compound that, when administered to a mammal for treating a state, disorder, disease or condition, is sufficient to effect such treatment. The effective amount or therapeutically effective amount will vary depending on the compound, the disease and its severity, and the age, weight, physical condition and responsiveness of the individual to be treated. In all cases, however, it is understood that therapeutically active component is ionic gallium.

“Delivering” and “administering” means providing a therapeutically effective amount of an active ingredient to a particular location or locations within a host causing a therapeutically effective blood concentration of the active ingredient at the particular location or locations. This can be accomplished, e.g., by local or by systemic administration of the active ingredient to the host.

The term “coadministration” encompasses administration of a first and second agent (e.g., gallium and a compound represented by structural formula I) in an essentially simultaneous manner, such as in a single dosage form, e.g., a capsule or tablet having a fixed ratio of first and second amounts, or in multiple dosage forms for each. The agents can be administered in a sequential manner in either order. When coadministration involves the separate administration of each agent, the agents are administered sufficiently close in time to have the desired effect (e.g., complex formation).

“Pharmaceutical grade” is used herein to denote that a compound or composition meets the standards regarding biologically active and/or inactive agents set by the various national bodies which regulate quality and reproducibility of pharmaceutical products. Such standards may include purity, activity, stability, etc. Generally, “pharmaceutical grade” means that the compound or composition is suitable for administration at therapeutic doses to a living organism without causing unwanted side effects.

“Pharmaceutically acceptable” means those active agents, salts and esters, and excipients which are, within the scope of sound medical judgment, suitable for use in contact with the tissues of humans and lower animals without undue toxicity, irritation, allergic response and the like, commensurate with a reasonable benefit/risk ratio, and effective for their intended use.

“A condition or disease characterized by excessive bone resorption” means any state, disorder, disease or condition which is characterized at least in part by excessive calcium resorption from bone, including, but not limited to, any cancer with metastatic lesions in bone, hypercalcemia, osteoporosis, osteopenia, Paget's disease, malignant bone disease, and hyperparathyroidism, as well as infections.

The term “composition” is intended to encompass a product comprising the specified ingredients in the specified amounts, as well as any product which results, directly or indirectly, from combination of the specified ingredients in the specified amounts.

One aspect of the present invention is directed to a method for replacing one or more anionic counterions of a gallium salt with one or more citrate ions comprising reacting the gallium salt with a citrate salt in an aqueous solution, and recovering the resulting gallium citrate in which one or more anionic counterions have been replaced with one or more citrate ions.

Applicants have found that the use of a citrate salt in the preparation of gallium citrate, in contrast to the prior art use of citric acid, allows the reaction to proceed at a faster rate with greater yield. In this regard, unlike prior art methods, no neutralization of the gallium/citrate mixture is required prior to recovery. Also, no special steps, such as ion exchange, are required in the process. The recovered product, unlike prior art forms, is also more suitable for immediate incorporation into pharmaceutical formulation processes to yield the final drug product, giving far greater efficiency.

Any gallium salt can be used in the preparative method, such as gallium chloride, gallium sulfate or gallium iodide, but preferably the gallium salt is gallium nitrate, such as Ga(NO₃)₃.9H₂O, which is the active pharmaceutical ingredient found in Ganite®.

Similarly, any citrate salt can be used in the preparative method, such as potassium citrate and sodium citrate, but preferably the citrate salt is ammonium citrate.

Generally, the gallium salt is dissolved in an aqueous solution and mixed with the citrate salt, which precipitates the dissolved gallium. The gallium is preferably at a concentration of about 0.1 M to saturation, more preferably about 0.75-1.25 M, most preferably about 1.0 M, while the citrate is preferably at a concentration of about 0.1 M to saturation, more preferably about 0.5-4 M, most preferably about 2-3 M. The gallium salt counteranion is stripped from the precipitated gallium by e.g., filtration. The gallium citrate can be washed with simple pharmaceutical solvents, pure water, or mixtures thereof to further reduce the amount of stripped counteranion. Suitable solvents include those on the Class 3 solvent list. If necessary, the gallium citrate can be recovered from the initial reaction by liquid/liquid extractions using solvents such as, but not limited to, butyl acetate, ethyl acetate, ethyl ether, ethyl formate, isopropyl acetate, and methyl ketone. The recovered material can be optionally dried by, e.g., oven heating or rotary evaporation.

Depending on the gallium salt and citrate salt used in the preparative method, the resulting gallium citrate may have low amounts of one or more pharmaceutically acceptable cations bonded thereto. As such, the recovered gallium citrate will have the general formula I:

[Cat]ₓGa(Cit)_{y}[An]_{z}.nH₂O I

wherein [Cat] is a pharmaceutically acceptable cation, (Cit) is citrate wherein 0-3 of the carboxyl groups are optionally protonated, [An] is a pharmaceutically acceptable anion, x is 0-2, y is 1-6, z is 0-2, and n is 0-2 or n is 0-6. In a preferred embodiment, x is 0, y is 2, z is 0 and n is 0. (Cit) in which no carboxyl group is protonated is C₆H₅O₇.

In a preferred embodiment, the starting gallium salt in the preparative method is gallium nitrate, and the citrate salt is ammonium citrate. Accordingly, the recovered gallium citrate will have the general formula II:

[NH₄]ₓGa(Cit)_{y}[NO₃]_{z}.nH₂O II

wherein (Cit) is as above, x is 0-2, y is 1-6, z is 0-2, and n is 0-2 or n is 0-6. In a preferred embodiment, x is 0, y is 2, z is 0 and n is 0.

Applicants have found that the gallium citrate made by the process disclosed herein has lower amounts of undesirable complexed ions, such as ammonium, sodium, potassium, and nitrate ions, compared to prior art gallium citrate preparations, and thus is more appropriate for administration to living organisms. Accordingly, another aspect of the present invention is directed to pharmaceutical grade gallium citrate. In a preferred embodiment, the pharmaceutical grade gallium citrate has the formula Ga(C₆H₅O₇)₂. This combines the lowest possible size with the highest delivery of gallium.

The pharmaceutical grade gallium citrate finds utility in the treatment of diseases and conditions characterized by excessive bone resorption, such as, e.g., cancer, hypercalcemia, osteoporosis, osteopenia, Paget's disease, malignant bone disease, and bone degeneration due to hyperparathyroidism, as well as infections

In the treatment of these and other diseases, a therapeutically effective amount of gallium citrate is administered to a patient in need of such treatment. The gallium is usually administered to a patient in the form of a pharmaceutical composition containing one or more pharmaceutically acceptable excipients. As gallium citrate is somewhat hydrophobic due to the bonding of the polycarboxylic acid groups of the citrate moieties with gallium, the gallium citrate preparation is expected to interact well with the gut, and thus is particularly suited to oral delivery. However, the gallium citrate compositions of the present invention can also be administered by other routes including, but not limited to, e.g., nasal, buccal, sublingual, intranasal, rectal, topical, transdermal, subcutaneous, intravenous, intraarterial, intramuscular, intraventricular, intraarticular, intraperitoneal, intrapleural, and intrathecal.

The particular pharmaceutical excipient(s) chosen, as well as the form of the composition, will depend at least in part on the desired administration route. Examples of pharmaceutically acceptable excipients and methods of manufacture for various compositions are well known in the art and may be found in, e.g., Remington's Pharmaceutical Sciences, (Gennaro ed., 20th ed. 2000).

For example, when used for oral administration, which is preferred, the gallium citrate composition will preferably be in solid tablet, capsule, caplet or dragee form incorporating one or more solid excipients, such as, e.g., starch, lactose, dextran, magnesium strearate, microcrystalline cellulose, crospovidone, sodium starch glycolate, silicon dioxide, etc.

In this regard, the gallium citrate made by the process disclosed herein is in the form of a fine powder. As such, no crushing, milling or grinding is necessary to prepare the material for tableting. The gallium citrate in the form of a wet cake can simply be mixed with desired excipients and granulated or directly compressed into tablets.

A tableted gallium citrate will preferably have an oral bioavailability profile (e.g., Peak [Ga]ₚₗₐₛₘₐ; Time to Peak [Ga]ₚₗₐₛₘₐ; etc.) similar to that for IV administration of an equal amount of gallium. Gallium citrate tablets containing 30 mg gallium may provide peak [Ga]ₚₗₐₛₘₐ of about 0.1 to 10 μg/mL or about 1,000 to 3,000 ng/mL at a time of between about 30 and 60 minutes. Tablets may also contain less than about 30 mg gallium, up to about 150 mg gallium or up to about 300 mg gallium. These tablets may provide steady-state [Ga]ₚₗₐₛₘₐ of between about 0.2 and 3.0 μg/mL or between about 1,000 and 40,000 ng/mL at times between about 15 to 120 minutes. In preferred embodiments, the tablets contain ≦100 mg nitrate/tablet or ≦60 mg nitrate/tablet.

In another preferred embodiment, the gallium citrate composition is formulated for pulmonary delivery. Such formulations are particularly useful for treating bacterial infections of the tissues and lungs formation. See, e.g., U.S. Pat. No. 5,997,912, U.S. Patent Publication No. 2006/0018945, and Kaneko et al., J. Clin. Invest. 117:877-888 (2007).

However, as shown below in the Examples, gallium need not be delivered by intra-pulmonary or inhalation administration to achieve concentrations in lung or sputum that may be therapeutic against bacterial infections. Systemic administration of ionic gallium, e.g., by intravenous, subcutaneous or oral administration, can achieve gallium concentrations in sputum sufficiently high to kill antibiotic-resistant Pseudomonas organisms in vivo. As shown in the Examples, intravenous administration is clearly effective in this regard, and by analogy other methods of systemic administration that can achieve comparable plasma concentrations should be equally therapeutic. As such, systemic administration of ionic gallium-containing compounds, such as gallium nitrate, gallium citrate and combinations thereof (e.g., Ganite ), are contemplated for the treatment of infections resulting from both gram-negative and gram-positive bacteria (both lung and non-lung associated), as well as for specific infectious conditions, such as, e.g., sepsis, pneumonia, osteomyelitis, etc.

The gallium citrate composition can also be in the form of a solution, suspension or emulsion incorporating a liquid excipient, such as, e.g., water, propylene glycol, polyethylene glycol, sorbitol, maltitol, sucrose or a pharmaceutically acceptable buffer, such as phosphate or carbonate buffer.

The pharmaceutical composition, particularly those for oral delivery, may also contain an oral delivery excipient capable of complexing gallium (i.e., functional excipient). Such a complexing agent may bind the gallium citrate, thereby increasing its bioavailability. Suitable complexing agents are those disclosed in U.S. Pat. No. 7,354,952, the content of which is incorporated herein in its entirety. A preferred complexing agent is represented by structural formula III:

2-OH—Ar—CR₁—NR₂—R₃—COOH III
wherein 2-OH—Ar is an optionally substituted 2-hydroxyaryl;
R₁ is —OH or ═O;
R₂ is hydrogen, hydroxyl or an optionally substituted C₁-C₄ alkyl, C₁-C₄ alkoxy, or C₂-C₄ alkenyl; and
R₃ is an optionally substituted aryl, heteroaryl, cycloalkyl, heterocyclyl, C₁-C₂₄ alkyl, C₂-C₂₀ alkenyl, C₂-C₂₀ alkynl, C₁-C₁₀ alkylaryl, C₁-C₁₀ arylalkyl, C₂-C₁₀ alkenylaryl, C₂-C₁₀ arylalkenyl, C₂-C₁₀ alkynylaryl or C₂-C₁₀ arylalkynyl,
which optionally is interrupted by O, N, S or any combination thereof.
   Specific nonlimiting examples of complexing agents according to structural formula III are N-(8-[2-hydroxybenzoyl]amino)caprylate (SNAC) and N-(10-[2-hydroxybenzoyl]amino decanoic acid (SNAD).

When it is desired to use functional carriers, such as those disclosed U.S. Pat. No. 7,354,952, in combination with the gallium compounds according to the present invention, the required amount of such carriers can be reduced as compared to their use with Ga(NO₃)₃ compounds because of the higher bioavailability of the gallium compounds of the present invention.

Although complexing agents may be used, they are not necessary. The nature of the gallium preparations described herein allows them to be formulated for therapeutic administration without the need for functional carriers or excipients. As such, pharmaceutical compositions, such as tablets, may be formulated using only GRAS, EAFUS or other FDA approved excipients.

The pharmaceutical compositions described herein generally comprise from about 1 to about 99 weight percent of gallium citrate. Preferably, when a pharmaceutically acceptable excipient is employed in the pharmaceutical compositions of the present invention, the compositions contain from about 1 to about 99 weight percent of the excipient. When the compositions also contain a complexing agent, the agent is generally present at no more than about 98 weight percent of the composition.

Doses are selected to provide pharmaceutically active plasma gallium concentrations for the treatment of excessive resorption of calcium from bone (e.g., arising from cancer, hypercalcemia, osteoporosis, osteopenia, Paget's disease, infections, etc.), which is established to be about 0.1-20.0 μg/ml, preferably about 0.5-2.0 μg/ml. Such blood levels may be achieved by administering about 0.1-2.0 grams of gallium daily.

For the treatment of various forms of cancer, including cancer-related hypercalcemia, gallium is typically administered in the range from about 0.1 mg/kg/day to about 15 mg/kg/day, preferably from about 0.25 mg/kg/day to about 7.5 mg/kg/day, from which the administration of the gallium citrate compositions of the present invention can be extrapolated. Such doses may be administered as a single unit dose or in a number of smaller doses.

Other dosage regimens for gallium are well known to those skilled in the art (see, e.g., Physician's Desk Reference, 58^{th} ed. (2004)). Dosages may be varied depending upon the requirements of the patient and the severity of the condition being treated. The amount and frequency of administration will be regulated according to the judgment of the attending clinician considering such factors as age, condition and size of the patient as well as severity of the symptoms being treated.

Specific embodiments according to the methods of the present invention will now be described in the following examples. The examples are illustrative only, and are not intended to limit the remainder of the disclosure in any way.

### EXAMPLES

### Example 1

One to six equivalents of ammonium citrate was incrementally added to an aqueous solution containing one equivalent of Ga(NO₃)₃.9H₂O at room temperature. At this point, spontaneous crystallization occurred rapidly. The mixture, which forms a precipitate (fine colloid-like material), was stirred at room temperature to a pH of about 3-4.5. The reaction is allowed to cool to room temperature. The nitrate in solution was stripped from the precipitated gallium citrate by filtration through Whatman 3M filter paper. The gallium citrate was then washed sequentially with 50% isopropanol in water and 190 proof ethanol and rotary evaporated at 60-100° C. under high vacuum to yield a gallium citrate preparation in the form a fine powder.

### Example 2

Prior to drying, two independent batches of gallium citrate from Example 1 were mixed separately with standard excipients, wet granulated and compressed into two separate lots of ˜725 mg tablets containing 30 mg gallium. The tablets from each of these lots contained about 22 μg of nitrate. For comparison, two batches of Ga(NO₃)₃.9H₂O (Ganite®) were formulated into separate lots of 750 mg tablets containing 30 mg gallium. The tablets from each of these lots contained about 80 mg nitrate.

### Example 3

The properties of each of the tablets from Example 2 were compared directly in single-dose dog studies. Tablets were orally administered to sets of four dogs and plasma levels of gallium determined at various time points following administration. Results for the two gallium citrate batches and the better performing gallium nitrate batch (clinical prototype) are shown below in Table 1.

TABLE 1

| | Gallium | Gallium Citrate | Gallium Citrate |
|---|---|---|---|
| | Nitrate | Batch 1 | Batch 2 |
| Peak [Ga]ₚₗₐₛₘₐ ng/mL, mean | 1,640 | 1,825 | 1,700 |
| No. Dogs ≧ 2000 ng/mL | 2 | 1 | 3 |
| No. Dogs ≧ 1,400 ng/mL | 3 | 3 | 3 |
| No. Dogs ≦ 1,400 ng/mL | 1 | 1 | 1 |
| Time to Peak [Ga]ₚₗₐₛₘₐ, min, | 195 | 100 | 113 |
| mean | | | |
| No. Dogs ≧ 180 min to Peak | 3 | 1 | 1 |
| [Ga]ₚₗₐₛₘₐ | | | |

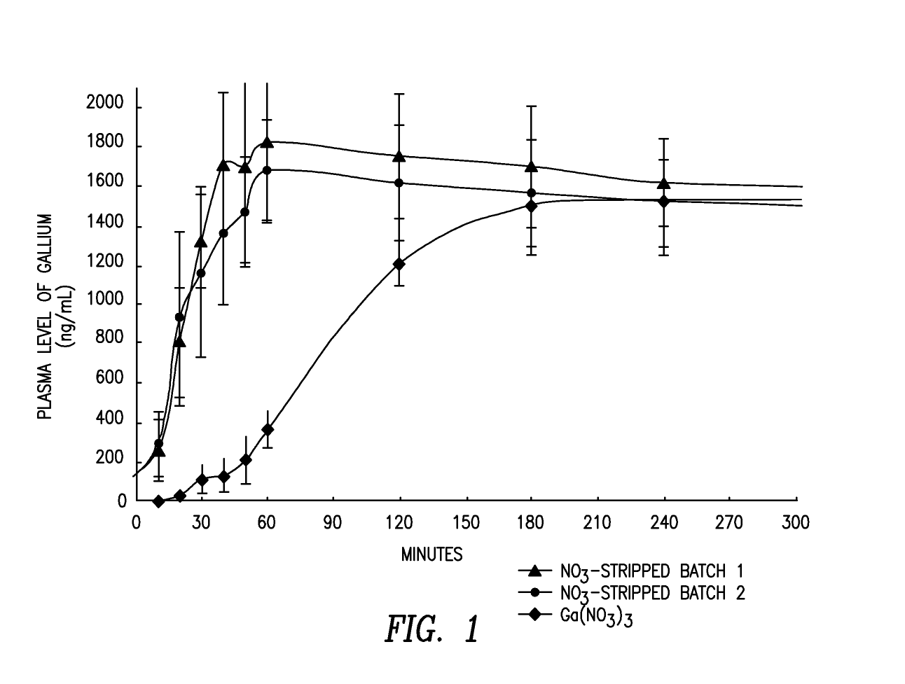

The bioavailability of the two lots of gallium citrate tablets and the better performing lot of the gallium nitrate (clinical prototype) tablets following oral administration to dogs is graphically depicted in FIG. 1. As clearly shown, oral absorption of gallium (Tmax≈30-60 min) was significantly more rapid from each of the gallium citrate tablets than from the gallium nitrate tablet (Tmax≈90-180 min). In this respect, oral administration of gallium citrate mimics IV administration of gallium nitrate. See Leyland-Jones, Seminars in Oncology, 18(4) (1991). Furthermore, both of the gallium citrate lots exhibited remarkably similar absorption profiles. For example, the lots generally exhibit a difference in Tmax of less than about 20%, preferably less than about 10%, more preferably less than about 5%, most preferably less than about 1%. In contrast, the lower performing lot of gallium nitrate tablets (Phase I clinical lot) exhibited ˜40% less peak [Ga]ₚₗₐₛₘₐ than the higher performing lot.

### Example 4

A patient with long-standing cystic fibrosis and a pulmonary infection with Pseudomonas aeruginosa that had proved highly resistant to all types of antibiotics was treated with several infusions of gallium citrate-nitrate intravenously at various dose levels suitable for human use without causing significant side-effects. During one such treatment, this patient received an infusion of gallium citrate-nitrate over 5 days at a dose of 240 mg/day, or approximately 150 mg/m²/day, and the concentration of ionic gallium in a deep-cough sputum specimen was then measured. The gallium concentration in the sputum was 3.2 μg/ml, or approximately 46 μmol/L, which is a concentration that is adequate to kill resistant Pseudomonas organisms in vivo. See Kaneko et al., J. Clin. Invest. 117:877-888 (2007). These data demonstrate that highly effective anti-bacterial concentrations of ionic gallium can be attained in sputum using systemic administration, such as intravenous, subcutaneous or oral treatment, and that intra-pulmonary administration, such as inhalation or nebulization, which is inconvenient and known to be potentially toxic to pulmonary tissues, is not required for such activity.

### Example 5

Beginning about 1 month later, the same patient from Example 4 was given additional infusions of gallium citrate-nitrate at doses of 200 mg/m²/day over 5 days, and the concentration of ionic gallium in deep-cough sputum specimens was again determined. The data presented in Table 2 below show that one month after the administration described in Example 4, the concentration of ionic gallium in lung sputum was still 0.32 μg/mL. From these data, it can be confidently concluded that gallium is retained in sputum for prolonged periods after systemic treatment. In addition, these data show that gallium is preferentially concentrated in sputum following systemic administration at levels that exceed plasma concentrations by roughly 2-fold. Further, these data show that sputum/lung represents a depot for gallium deposition, since gallium concentrations in sputum actually increase over time even though systemic administration has ended and plasma concentrations are progressively dropping. The concentrations at days 5-11 are high, and even after cessation of treatment they remain levels that are believed to be therapeutic in vivo.

TABLE 2

| | | | |
|---|---|---|---|
| | | | Result |
| Dose (mg/m²/d) | Time Collected | Source | (μg/mL) |

Treatment Number One

| | | | |
|---|---|---|---|
| 150 (end of infusion) | Day 5 | Sputum | 2.22 |

Treatment Number Two

| | | | |
|---|---|---|---|
| | Baseline (Time 0) | Sputum | 0.32 |
| 200 (end of infusion) | Day 5 | Sputum | 2.13 |
| 200 (end of infusion) | Day 5 (verification repeat) | Sputum | 2.65 |
| 200 (end of infusion) | Day 5 (verification repeat) | Plasma | 1.35 |
| 200 (end of infusion) | Day 5 20:00 h | Whole | 1.64 |
| | | Blood | |
| Post-Infusion | Day 6 | Sputum | 1.83 |
| | Day 7 | Sputum | 2.04 |
| | Day 9 | Sputum | 4.35 |
| | Day 11 | Sputum | 4.78 |

Although the invention has been described with reference to gallium citrate, other forms of gallium complexes can also be prepared using the methods disclosed herein. For example, instead of citrate, the gallium can be reacted with any mono-, di-, tri-, or tetra-carboxylic acid, and the resulting gallium complex recovered as described above. In this respect, the gallium complex will have the general formula IV:

[Cat]ₓGaL_{y}[An]_{z}.nH₂O IV

wherein [Cat] is a pharmaceutically acceptable cation, L is a pharmaceutically acceptable carboxylic acid, [An] is a pharmaceutically acceptable anion, x is 0-2, y is 1-6, z is 0-2, and n is 0-2 or n is 0-6.

Any pharmaceutically acceptable natural or unnatural carboxylic acid can be used in the reaction, but carboxylic acid is preferably a polycarboxylic acid from the Krebs cycle, such as citrate (already described herein), cis-aconitate, isocitrate, oxalosuccinate, alpha-ketoglutarate, succinate, fumarate, malate and oxaloacetate. Tartrate and citramalate can also be used. In this fashion, gallium tartrate has been prepared tartrate and gallium nitrate. For naturally occurring carboxylic acids that have a stereocenter, e.g., L-isocitrate, L-tartrate, etc., the physiological L-form, the non-physiological D-form, or a mixture of the L- and D-forms can be used, e.g., DL-isocitrate, DL-tartrate, etc.

In addition, the carboxylic acid can be derivatized to produce more hydrophobic molecules. In this respect, the L in general formula IV above will be a derivatized pharmaceutically acceptable carboxylic acid.

For example, the carboxylic acid can be derivatized by esterification of one or more of the carboxylate groups. The alkyl chains of the ester groups can include chains of one (methyl ester), two (ethyl ester), three (propyl ester), four (butyl ester, and longer, although even-numbered carbon chains are preferred. Examples of derivatized carboxylic acids include, but are not limited to, trimethylcitrate, triethylcitrate, tributylcitrate, dimethyl tartrate and diethyl tartrate. In this fashion, gallium tributylcitrate has been prepared from tributylcitrate and gallium nitrate.

Increasing the hydrophobicity of the gallium complex allows the therapeutic delivery of gallium directly, with no carrier, complexing agent, or functional excipient, or increases the therapeutic performance in the presence of these formulation components. As such, tablets incorporating these gallium complexes would be significantly reduced in size, thereby increasing patient compliance while reducing unwanted side-effects. For example, daily tablets for osteoporosis and other chronic diseases could be about 750-800 mg total weight, up to about 1000 mg, but would preferably be about 75-250 mg total weight.

In addition, more efficient delivery of gallium complexes allows therapeutic delivery for direct anti-tumor activity. In this respect, tablets could be about 1000-1200 mg total weight, with high enough gallium content (e.g., about 100 to 500 mg) to be used as a direct component of a chemotherapy regimen and thereby replace Ganite® (Ga(NO₃)₃.9H₂O). Intravenous Ganite® has been given as part of a Phase I clinical trial in non-Hodgkin's lymphoma in the GARD study.

The carboxylic acid L in general formula IV can also be derivatized by esterification of one or more of the carboxylate groups to produce more hydrophilic molecules. For example, L can be derivatized with small O— and C-based hydrophilic groups, such as polycarboxylates, polyhydroxylates, polyethers, etc. An example of a suitable group is PEG. Polymers of L in general formula IV (e.g., polycitrate) can also be used to increase the hydrophilicity of the complexes.

All publications cited in the specification, both patent and non-patent publications, are indicative of the level of skill of those skilled in the art to which this invention pertains. All these publications are herein fully incorporated by reference to the same extent as if each individual publication were specifically and individually indicated as being incorporated by reference.

What is claimed:

1. A method of treating a patient having a bacterial infection comprising systemically administering to said patient a compound in an amount sufficient to treat said infection, wherein the compound has the general formula IV:
[Cat]ₓGaL_{y}[An]_{z}.nH₂O IV
wherein [Cat] is a pharmaceutically acceptable cation, L is a pharmaceutically acceptable polycarboxylic acid, [An] is a pharmaceutically acceptable anion, x is 0-2, y is 1-6, z is 0-2, and n is 0-2 or n is 0-6.

2. The method of claim 1 wherein the compound is administered by a route selected from the group consisting of orally, intranasally, transbuccally, sublingually, intrarectally, or by subcutaneous, intravenous, intraarterial, intramuscular, intraventricular, intraarticular, intraperitoneal, intrapleural, or intrathecal injection or installation.

3. The method of claim 1, wherein the polycarboxylic acid is selected from the group consisting of citrate, cis-aconitate, isocitrate, oxalosuccinate, alpha-ketoglutarate, succinate, fumarate, malate, oxaloacetate, tartrate, citramalate, trimethylcitrate, triethylcitrate, tributylcitrate, dimethyl tartrate and diethyl tartrate.

4. The method of claim 1, wherein the compound is gallium citrate.

5. The method of claim 4 wherein the gallium citrate has the general formula II:
[NH₄]ₓGa(Cit)_{y}[NO₃]_{z}.nH₂O II.

6. The method of claim 5 wherein the gallium citrate has the formula Ga(C₆H₅O₇)₂.

7. The method of claim 1, wherein the compound is gallium tributylcitrate.

8. The method of claim 1, wherein the patient is infected with a gram-negative bacterium.

9. The method of claim 8, wherein the bacterium is a member of the genus Pseudomonas.

10. The method of claim 9, wherein the infection is pneumonia.

11. The method of claim 1, wherein the patient is administered a dose of at least about 50 mg/day of the compound.

12. The method of claim 11, wherein the patient is infused with the compound over a period of from about 1-5 days.

13. The method of claim 12, wherein the infusion provides a dose of approximately 150-200 mg/m²/day.

14. The method of claim 12, wherein the infusion provides a gallium concentration in sputum of about 2.13-2.65 μg/mL.

15. The method of claim 12, wherein the infusion provides a gallium concentration in sputum of at least about 3.0 μg/mL.

16. The method of claim 11, wherein the compound is administered by oral administration or pulmonary administration.

17. A method for achieving preferentially higher concentrations of gallium in the lung and sputum of a patient as compared to concentration of gallium in plasma comprising systemically administering to the patient a compound having the general formula IV:
[Cat]ₓGaL_{y}[An]_{z}.nH₂O IV
wherein [Cat] is a pharmaceutically acceptable cation, L is a pharmaceutically acceptable polycarboxylic acid, [An] is a pharmaceutically acceptable anion, x is 0-2, y is 1-6, z is 0-2, and n is 0-2 or n is 0-6.

18. The method of claim 17, wherein the patient is infected with a bacterium from the genus Pseudomonas.

19. The method of claim 17, wherein the compound is administered by a route selected from the group consisting of orally, intranasally, transbuccally, sublingually, intrarectally, or by subcutaneous, intravenous, intraarterial, intramuscular, intraventricular, intraarticular, intraperitoneal, intrapleural, or intrathecal injection or installation.

20. The method of claim 17, wherein the compound is gallium citrate or gallium tributylcitrate.
